(19) United States
(12) Patent Application Publication
(10) Pub. No.: US 2011/0082434 A1
(43) Pub. Date: Apr. 7, 2011

(54) SURGICAL COVER FOR HUMAN LIMB

(76) Inventor: Jerry P. SAGER, Warren, NJ (US)

(21) Appl. No.: 12/574,745

(22) Filed: Oct. 7, 2009

(51) Int. Cl. A61F 13/45; A61F 13/02

(52) U.S. Cl. 604/385.01

(57) ABSTRACT

The disclosure relates to a surgical cover for a human limb, such as a hand or a foot. The surgical cover includes an inner adhesive seal, an interior absorbent strip and an exterior adhesive seal strip. The combination of these three elements, along with the waterproof character of the walls of the surgical cover, provide a surgical cover which is waterproof or water-resistant, while providing for some use of the limb by the patient.

### BACKGROUND OF THE DISCLOSURE

1. Field of the Disclosure

The present disclosure pertains to a waterproof or water-resistant glove or sock-like cover to protect a hand or a foot, typically after a surgical operation when it is necessary to keep the hand or foot clean and dry, while preferably maintaining at least some use of the hand or foot.

2. Description of the Prior Art

It is well-known that it is necessary to keep a surgical site clean and dry during a recuperation period. This can be somewhat problematic for a surgical site on a limb, such as a hand or a foot, in that the patient typically seeks to maintain at least some use of the hand or foot. Additionally, it can be difficult to keep the surgical site on a limb, such as a hand or foot, dry during showering or similar activities.

### OBJECTS AND SUMMARY OF THE DISCLOSURE

It is therefore an object of the present disclosure to provide a device for maintaining a surgical site on a hand or foot clean and dry during a recuperation period, particularly during showering or similar activities.

It is therefore a further object of the present disclosure to provide a device for maintaining a surgical site on a hand or foot clean and dry while maintaining at least some use of the hand or foot.

These and other objects are attained by providing a mitten-shaped or sock-shaped bag-like structure, typically made from a clear thermoplastic or similar material, which fits over the hand or foot. The bag-like structure includes, near the opening, an interior adhesive seal, an interior absorbent strip and an exterior adhesive seal strip.

### BRIEF DESCRIPTION OF THE DRAWINGS

Further objects and advantages of the invention will become apparent from the following description and from the accompanying drawing, wherein:

FIG. 1 is a plan view of an embodiment of the disclosure adapted for use with a human hand.

FIG. 2 is a plan view of the embodiment of FIG. 1, showing a bandaged human hand therein.

FIG. 3 is a perspective view of the interior adhesive seal with the backing strip in place.

FIG. 4 is a perspective view of the interior adhesive seal with the backing strip partially removed.

FIG. 5 is a perspective view of the mouth of the embodiment of FIG. 1.

FIG. 6 is a plan view of the mouth of an embodiment wherein the exterior adhesive seal strip extends above the mouth of the embodiment in order to seal to both the patient's wrist and the surgical cover.

FIGS. 7A-7D show a typical sequence of applying the embodiment of FIG. 1 to a human hand.

FIGS. 8A-8C illustrate the sequence for the fastening of the exterior adhesive seal strip during the initial manufacturing of the embodiment of the disclosure.

FIG. 9 is a plan view of an embodiment of the disclosure adapted for use with a human foot.

### DETAILED DESCRIPTION OF THE PREFERRED EMBODIMENTS

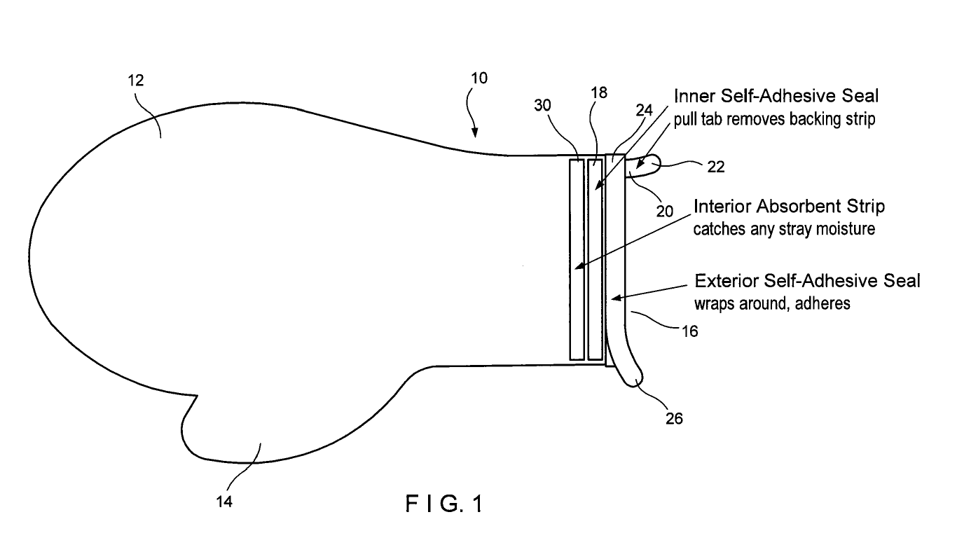

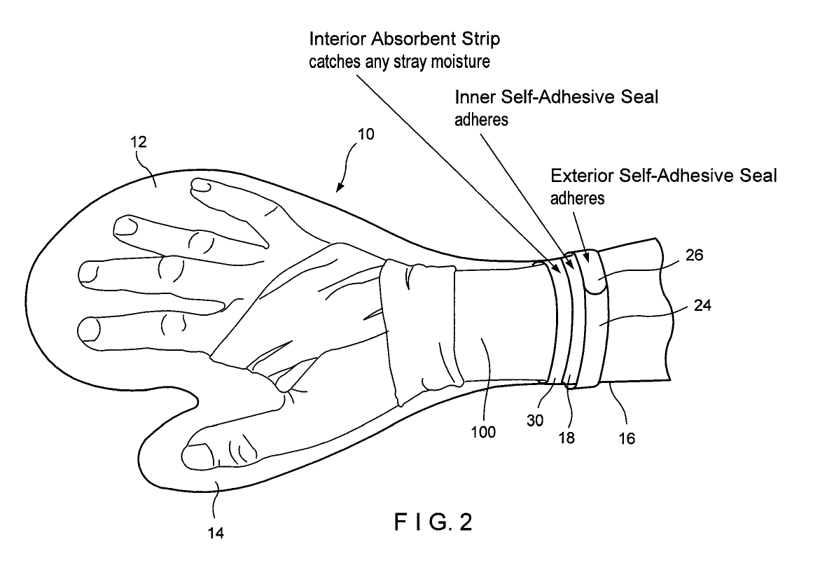

Referring now to the drawings in detail wherein like numerals indicate like elements throughout the several views, one sees that FIG. 1 illustrates an embodiment of the present disclosure, a surgical cover 10 adapted for protecting a surgical site on a human hand. FIG. 2 shows a patient's bandaged hand 100 inserted therein. The surgical cover 10 is generally shaped like a mitten with a volume therein including a larger section 12 for protecting the four fingers and a smaller section 14, protruding from a side of larger section 12, for protecting the thumb. Surgical cover 10 is typically formed as a bag-like structure with walls formed from a transparent polymeric or plastic material, such as, but no limited to, polyethylene. Those skilled in the art may recognize a range of equivalents after review of the present disclosure.

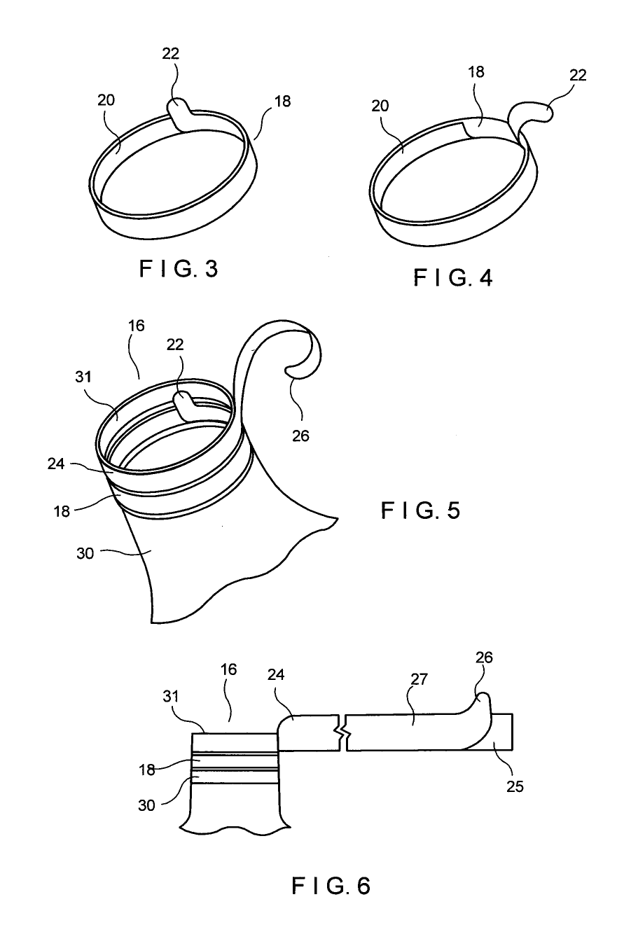

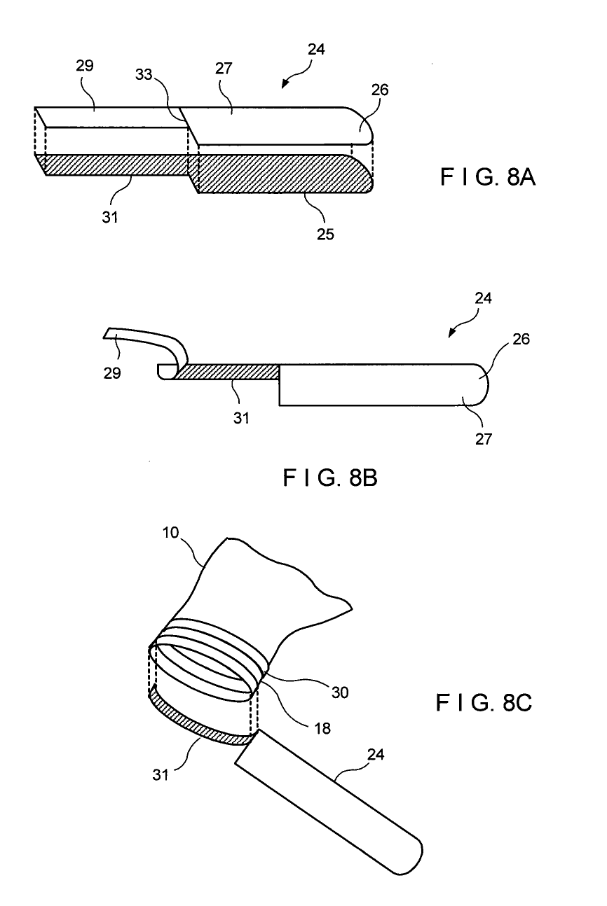

As shown in FIG. 1 and further illustrated in FIG. 5, surgical cover 10 further includes a mouth 16 for insertion of the hand therethrough. Immediately inwardly adjacent from the mouth 16, and on the interior surface thereof, is formed interior adhesive seal 18, formed in a ring shape, with a backing strip 20 terminating in a backing strip pull tab 22 for the manual removal of the backing strip 20. Interior adhesive seal 18 is illustrated in further detail in FIGS. 3 and 4. Likewise, immediately inwardly adjacent from the mouth 16, and on the exterior surface thereof, is formed exterior adhesive seal strip 24. Exterior adhesive seal strip 24 includes a first adhesive surface 25, of somewhat increased width, and a second adhesive surface 31, of somewhat reduced width. As shown in FIGS. 8A-8C, second adhesive surface 31 is typically applied during the manufacturing process by removing second backing paper portion 29 (typically initially joined to first backing paper portion 27 along a line of weakness 33) and placing second adhesive surface 31 against the surgical cover 10. The user thereby receives surgical cover 10 with first adhesive surface 25, covered with first backing paper portion 27, extending from the surgical cover 10.

Immediately inwardly adjacent from the inner adhesive seal 18 and likewise on the interior surface of mouth 16 is interior absorbent strip 30. Interior absorbent strip 30 is typically formed from cloth material, such as surgical absorbent material, in order to trap any moisture which may pass the interior adhesive seal 18.

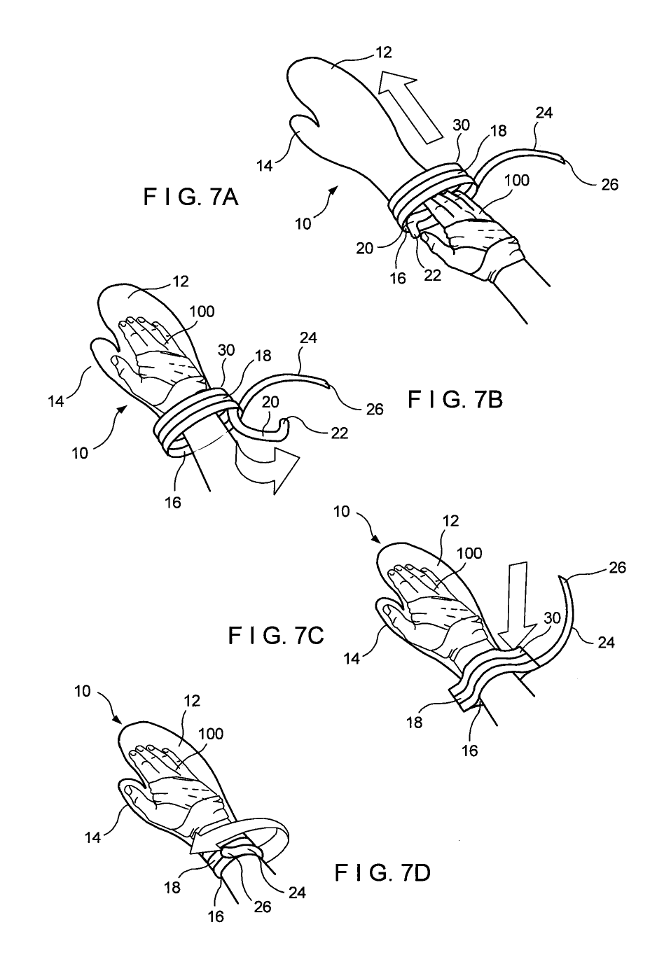

A typical sequence of applying surgical cover 10 is shown in FIGS. 7A-7D. First backing paper portion 27 is removed from the exterior adhesive seal strip 24 by use of pull tab 26 as shown in FIG. 7A. The patient's hand 100, typically including a surgical site, is inserted into surgical cover 10 through mouth 16 so that the patient's thumb aligns with smaller section 14 of the surgical cover as shown in FIG. 7B. The backing strip 20 is removed from interior adhesive seal 18 by the user pulling pull tab 22 thereby exposing an adhesive surface as likewise shown in FIG. 7B. The adhesive surface of interior adhesive seal 18 is pushed around the wrist of the patient to form a tight seal as shown in FIG. 7C. Exterior adhesive seal strip 24 is then wrapped tightly around the exterior surface adjacent to the mouth 16 so as to adhesively engage both the surgical cover 10 and the patient's wrist and to press the surgical cover 10 tightly against the patient's wrist thereby forming or contributing to a watertight or water-resistant configuration as shown in FIG. 7D.

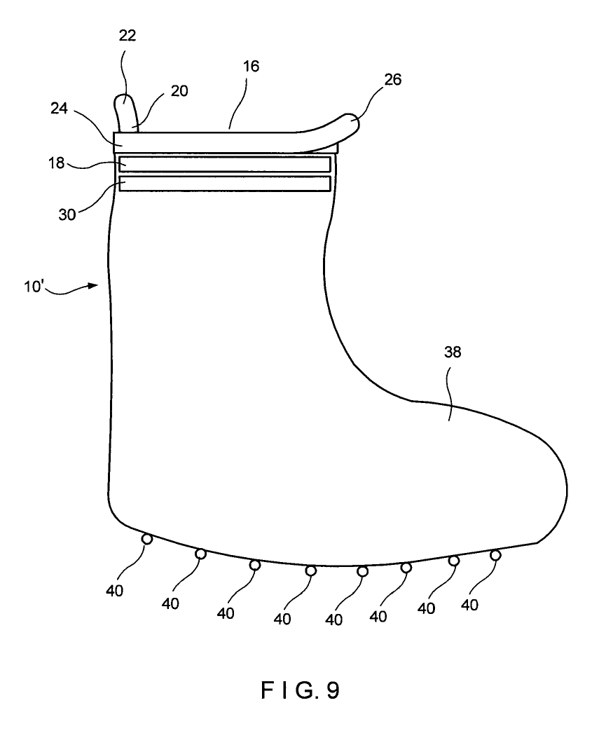

FIG. 9 illustrates an embodiment of surgical cover 10′ which is adapted and sized for the human foot (not shown). Smaller section 14 (adapted to a thumb) of surgical cover 10 is omitted but a perpendicular section 38 sized and configured to receive a human foot (which typically includes a surgical site) is provided. A human ankle is received within mouth 16. Additionally, rubber traction elements 40 are added at the bottom of surgical cover 10′ to aid in the traction required for walking after the surgical cover 10′ is applied. The interior adhesive seal 18, the exterior seal strip 24 and the interior absorbent strip 30 have substantially the same configuration in surgical cover 10′ as described for surgical cover 10.

Thus the several aforementioned objects and advantages are most effectively attained. Although preferred embodiments of the invention have been disclosed and described in detail herein, it should be understood that this invention is in no sense limited thereby and its scope is to be determined by that of the appended claims.

What is claimed is:

1. A surgical cover for a human limb, including:
walls forming a volume to receive the human limb;
a mouth formed in said walls through which a human limb is inserted into said volume;
an interior adhesive seal about an interior of said walls inwardly adjacent from said mouth;
an interior absorbent strip about an interior of said walls inwardly adjacent from said interior adhesive strip; and
an exterior adhesive seal strip extending from an exterior of the walls adjacent from the mouth.

2. The surgical cover of claim 1 wherein said walls are formed of transparent material.

3. The surgical cover of claim 1 wherein said walls are formed of plastic material.

4. The surgical cover of claim 1 wherein said walls are formed of polyethylene.

5. The surgical cover of claim 1 wherein said interior adhesive seal includes a backing strip which is removed by a user to expose an adhesive surface.

6. The surgical cover of claim 5 wherein said backing strip terminates a backing strip pull tab.

7. The surgical cover of claim 1 wherein said exterior adhesive seal strip terminates in a seal strip pull tab.

8. The surgical cover of claim 1 wherein said volume comprises a first portion for receiving a hand and a second portion, extending from said first portion, for receiving a thumb.

9. The surgical cover of claim 1 wherein said volume comprises a first portion for receiving an ankle and a second portion, extending substantially perpendicularly from said first portion, for receiving a foot.

10. The surgical cover of claim 9 further including traction elements formed on a bottom of the surgical cover.

11. The surgical cover of claim 10 wherein said traction elements are made of rubber.

12. The surgical cover of claim 1 wherein the interior absorbent strip is formed of cloth.

13. The surgical cover of claim 12 wherein the cloth is surgical absorbent material.
